(19) United States
(12) Patent Application Publication
(10) Pub. No.: US 2001/0049457 A1
(43) Pub. Date: Dec. 6, 2001

(54) Method of fluorinating a halogenated organic substance

(76) Inventor: Matthew D. Stephens, Arlington, MA (US)

Correspondence Address: IANDIORIO & TESKA, 260 BEAR HILL ROAD, WALTHAM, MA, 02451-1018, US

(21) Appl. No.: 09/752,349

(22) Filed: Dec. 29, 2000

Related U.S. Application Data
(60) Provisional application No. 60/173,575, filed on Dec. 29, 1999.

(51) Int. Cl. C07C 17/20

(52) U.S. Cl. 570/123

(57) ABSTRACT

A method of fluorinating a halogenated organic substance comprising the steps of: introducing a germanium fluoride compound to a halogenated organic substance at a temperature between 250 and 320° C. to increase the fluorine content of the halogenated organic substance, thereby forming a fluorinated organic substance and a halogenated germanium compound, and separating the fluorinated organic substance and the halogenated germanium compound.

### RELATED APPLICATIONS

This application claims priority from U.S. Provisional Patent Application Ser. No. 60/173,575 filed Dec. 29, 1999.

### FIELD OF INVENTION

This invention relates to a method of fluorinating a halogenated organic substance.

### BACKGROUND OF INVENTION

The fluorination of halogenated organic compounds has many uses, especially relating to pharmaceuticals and the environment. The fluorination of halogenated organic compounds is used to manufacture specialty chemicals, remove chlorine from chlorocarbons to produce fluorocarbons, to convert CFC stockpiles to useful perfluorocarbons, to produce new hydrofluorocarbon (HFC) refrigerants from chlorocarbons, to produce polytetrafluoroethylene (PTFE or Teflon), and surface fluorination of melt-processable chlorinated polymers.

There are many methods employed to introduce fluorine into inorganic and organic compounds. However, many of these methods are not suitable for the production of useful chemicals from halogenated organic compounds. For instance, elemental fluorine (F₂) can be used to introduce fluorine into compounds. Unfortunately, this substance is very reactive and tends to cause unwanted side reactions that lead to the rapid decomposition of desired products. In addition, the extreme reactivity of this reagent presents a significant obstacle to its use in many practical applications because of safety considerations. Thus, it is not practical in many instances to employ F₂ to introduce fluorine into halogenated organic compounds.

Other methods to introduce fluorine into compounds have been developed. For instance, HF, antimony fluorides, or high-valence transition metal fluorides can be used to introduce fluorine into compounds. However, in many instances, the conditions required for rapid reaction are too extreme to yield useful products that can be extracted from the process stream in an economical manner. Further, when mild conditions are employed, typical processes require a long residence time in the reactor. Because of these issues, typical fluorinated organic compounds such as PTFE (Teflon) are very expensive to produce relative to other industrial chemicals such as halogenated organics. This is unfortunate because fluorinated compounds possess properties that make them useful in a wide variety of applications including refrigeration, agriculture, medicine, metal production, and manufacturing.

One approach used to fluorinate halogenated organic compounds, and in particular chlorofluorocarbons, is to use fluorintaing agents with a catalyst, such as SbCl₅. However, reactions such as these are nonselective and have a long reaction time and low yield of product.

### SUMMARY OF THE INVENTION

It is therefore an object of this invention to provide a fluorinating agent for a halogenated organic compound that reacts quickly.

It is a further object of this invention to provide a fluorinating agent for a halogenated organic compound that reacts selectively.

It is a further object of this invention to provide a fluorinating agent for a halogenated organic compound that is cheaper than those fluorinating agents currently used.

It is a further object of this invention to provide a fluorinating agents for a halogenated organic compound that is less toxie than several of those fluorinating agents currently used.

The invention results from the realization that a truly effective method of fluorinating a halogenated organic compound can be achieved introducing a germanium fluoride compound to the halogenated organic compound to produce a fluorinated organic substance and a halogenated germanium compound.

This invention features a method of fluorinating a halogenated organic substance comprising the steps of introducing a germanium fluoride compound to a halogenated organic substance at a temperature between 250 and 320° C. to increase a fluoride content of the halogenated organic substance, thereby forming a fluorinated organic substance and a halogenated germanium compound, and separating the fluorinated organic substance and the halogenated germanium compound.

In a preferred embodiment, the germanium fluoride compound may be of the form of RGeFₓ. The germane fluoride compound may be halogenated. The germanium fluoride compound may be GeF₄. The germanium fluoride compound may include either a halogen or an organic substituent. The organic subsituent may include a hydrocarbon, a fluorinated hydrocarbon, or a perfluorocarbon. The halogenated organic substance may be chosen from the group consisting of CHCl₃, CCl₄, C₂Cl₆ and CH₃ CCl₃. The fluorinated organic substance may be CHF₃. The fluorinated organic substance may be CHF₂Cl. The introduction of the germanium fluoride and the halogenated organic substance may be done in the presence of a solvent.

This invention also features a method of fluorinating a halogenated organic substance comprising the steps introducing GeO₂ and UF₄ to CHCl₃ at a temperature between 250 and 320° C. to form CHF₃ and GeCl₄, and separating the CHF₃ and GeCl₄.

The introduction of the GeO₂ and UF₄ to the CHCl₃ may be done in the presence of a solvent.

This invention also features a method of fluorinating a halogenated organic substance comprising the steps of providing a halogenated organic substance chosen from the group consisting of CHCl₃, CCl₄, C₂Cl₆, and CH₃CCl₃, introducing a germanium fluoride compound to the halogenated organic substance at a temperature between 250 and 320° C. to increase the fluorine content of the halogenated organic substance, thereby forming a fluorinated organic substance and a halogenated germanium compound, and separating the fluorinated organic substance and the halogenated germanium compound.

In a preferred embodiment, the step of introducing a germanium fluoride compound may further include the step of introducing HF to a germanium fluoride compound to produce GeF₄. The step of introducing a germanium fluoride compound may further include the step of adding an inorganic fluorine compound to a halogenated germanium compound to increase the fluorine content of the halogenated germanium compound and to form a germanium fluoride compound. The introduction of the germanium fluoride to the halogenated organic substance may be done in the presence of a solvent.

This invention also features a method of fluorinating a halogenated organic substance comprising the steps of introducing an inorganic fluorine compound to a halogenated, germanium compound to increase the fluorine content of the halogenated germanium compound and to form a germanium fluoride compound, introducing the germanium fluoride compound to a halogenated organic substance at a temperature between 250 and 320° C. to increase the fluorine content of the halogenated organic substance, thereby forming a fluorinated organic substance and a halogenated germanium compound, and separating the fluorinated organic substance and the halogenated germanium compound.

In a preferred embodiment, the germanium fluoride compound may be of the form of RGeFₓ. The germane fluoride compound may be halogenated. The germanium fluoride compound may be GeF₄. The germanium fluoride compound may include either a halogen or organic substituent. The organic substituent may include a hydrocarbon, a fluorinated hydrocarbon, or a perfluorocarbon. The halogenated organic substance may be chosen from the group consisting of CHCl₃, CCl₄, C₂Cl₆ and CH₃CCl₃. The fluorinated organic substance may be CHF₃. The fluorinated organic substance may be CHF₂Cl. The introduction of the germanium fluoride and the halogenated organic substance may be done in the presence of a solvent. The inorganic fluorine compound may be chosen from the group consisting of HF, F₂, SbF₃, SbF₅, and the transition metal fluorides, including specifically AgF₂, CoF₅, UF₆ and UF₄. The inorganic fluorine compound may also be chosen from the group consisting of transition metal oxyfluorides, including specifically UO₂F₂.

### BRIEF DESCRIPTION OF THE DRAWINGS

Other objects, features and advantages will occur to those skilled in the art from the following description of a preferred embodiment and the accompanying drawings, in which:

FIG. 1 is a block diagram of the method of fluorinating a halogenated organic substance according to the present invention.

FIG. 2 is a block diagram of the method of fluorinating a halogenated organic substance according to a further embodiment of the present invention.

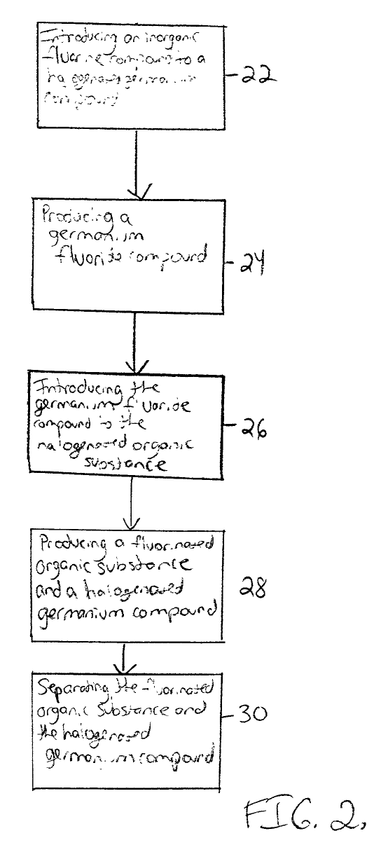

### PREFERRED EMBODIMENT

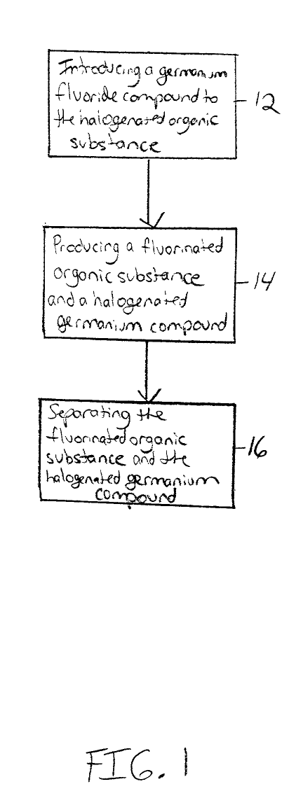

A germanium fluoride compound is reacted with a halogenated organic substance to increase the fluorine content of the halogenated organic substance, FIG. 1. First, the halogenated organic substance to be fluorinated is chosen. The germanium fluoride compound is then introduced to the halogenated organic substance 12. This mixture is preferably then subjected to temperatures in the range of 250-320° C., and more preferably between 275° and 320° C. Such temperatures are high enough to promote reactions without destroying the organic substance. The reaction of the germanium fluoride compound and the halogenated organic substance produces a fluorinated organic substance and a halogenated germanium compound 14. The fluorinated organic substance is then separated from the halogenated germanium compound 16, for example by gas separation or by washing.

The combination of the germanium fluoride compound and the halogenated organic substance can also occur in the presence of a solvent. The solvent can be introduced in a variety of manners, including introducing the germanium fluoride compound to a solvent and then introducing the mixture to the halogenated organic substance; introducing the germanium fluoride compound to a solvent, introducing the germanium fluoride compound compound and the halogenated organic substance to a solvent simultaneously. Examples of the type of solvent than can be used include diglyme, acentonitrile and diethyl ether.

A germanium fluoride compound is used to fluorinate the halogenated organic substance for its ability to react quickly yet selectively with halogenated organic substances. Additionally, germanium fluoride compounds possess the ability to quantitatively exchange fluorine for other halogens, such as chlorine.

Any germanium fluoride compound of the form RGeFₓ is suitable to fluorinate halogenated organic substances, in which R denotes the rest of the molecule, for instance, a halogen or organic substituent. One specific germanium fluoride compound found to be particularly useful in fluorinating halogenated organic substances is GeF₄. Although GeF₄ can be used to fluorinate halogenated organic substances in general, GeF₄ is particularly useful in fluorinating chlorocarbons (for example CHCl₃, CCl₄, C₂Cl₆ and CH₃CCl₃). GeF₄ is chosen for its ability to quantitatively exchange fluorine for chlorine from hyrocarbons to produce fluorocarbons. For example, GeF₄ can be reacted with chloroform in the following manner to produce fluoroform:

3GeF₄+4CHCl₃=3GeCl₄+4CHF₃

During the reaction of the GeF₄ with the chloroform, which results in the production of CHF₃, CHF₂Cl and CHCl₂F are produced at intermediate steps. The CHF₂Cl which is produced is also a very useful product. When reacting GeF₄ with any of the above chlorocarbons, the resulting fluorinated organic substance will be CHF₃, CHF₂Cl, CHCl₂F or a mixture of the substances. When reacting GeF₄ with a chlorofluorocarbon, such as dichlorodifluoromethane as shown below, the resulting fluorinated organic substance can also include CF₄.

GeF₄+2CCl₂F₂=GeCl₄+2CF₄

An alternative to introducing the germanium fluoride compound directly to the halogenated organic substance to be fluorinated is to generate the germanium fluoride compound “in situ”, or during the fluorination process. First, the halogenated organic substance to be fluorinated is selected. In an “in situ” fluorination process, an inorganic fluorine compound is introduced to a halogenated germanium compound 22. The reaction of the compounds increases the fluorine content of the halogenated germanium compound and forms a germanium fluoride compound 24. The germanium fluoride compound is then introduced to the halogenated organic substance to produce a fluorinated organic substance and a halogenated germanium compound 26. The fluorinated organic substance is then separated from the halogenated germanium compound 28, for example by gas separation or by washing.

For example, UF₄ and GeO₂ can be reacted to generate the germanium fluoride compound GeF₄. Specifically, as an example, 3.7 g of UF₄ and 1.2 g of GeO₂ can be used to produce GeF₄. As the GeF₄ is being generated, it is swept through tubing with 100 cm³/min of dry, CO₂-free air into a ploypropylene Erlenmeyer flask containing the halogenated germanium compound to be fluorinated; in this case 100 ml of CHCl₃. The CHCl₃ is subjected to temperatures in the range of 250-320° C. throughout the reaction. This reaction results in the fluorination of the CHCl₃ to produce fluorinated organic substances CHF₂Cl, CHCl₂F and CHF₃ and halogenated compound GeCl₄, which are then separated.

Although UF₄ is used as the inorganic fluorine compound in the above example, any inorganic fluorine compound can be used. Specifically, HF or F₂, antimony fluorides such as SbF₃ or SbF₅, transition metal fluorides including specifically AgF₂, CoF₅, UF₆ and UF₄, or transition metal oxyfluorides, including specially UO₂F₂ would be suitable.

Alternatively, GeF₄ could be produced by the decomposition of a hexafluorogermanate salt such as Na₂GeF₆ or BaGeF₆ and subsequently reacted with a halogenated organic substance.

Although specific features of the invention are shown in some drawings and not in others, this is for convenience only as each feature may be combined with any or all of the other features in accordance with the invention.

Other embodiments will occur to those skilled in the art and are within the following claims:

What is claimed is:

1. A method of fluorinating a halogenated organic substance comprising the steps of:
introducing a germanium fluoride compound to a halogenated organic substance at a temperature between 250 and 320° C. to increase the fluorine content of the halogenated organic substance, thereby forming a fluorinated organic substance and a halogenated germanium compound; and
separating the fluorinated organic substance and the halogenated germanium compound.

2. The method of fluorinating a halogenated organic substance of claim 1 in which the germanium fluoride compound is of the form of RGeFₓ.

3. The method of fluorinating a halogenated organic substance of claim 2 in which the germanium fluoride compound is halogenated.

4. The method of fluorinating a halogenated organic substance of claim 2 in which the germanium fluoride compound is GeF₄.

5. The method of fluorinating a halogenated organic substance of claim 2 in which the germanium fluoride compound includes a halogen or organic substituent.

6. The method of fluorinating a halogenated organic substance of claim 5 in which the organic substituent includes a hydrocarbon, a fluorinated hydrocarbon, or a perfluorocarbon.

7. The method of fluorinating a halogenated organic substance of claim 1 in which the halogenated organic substance is chosen from the group consisting of CHCl₃, CCl₄, C₂Cl₆ and CH₃CCl₃.

8. The method of fluorinating a halogenated organic substance of claim 1 in which the fluorinated organic substance is CHF₃.

9. The method of fluorinating a halogenated organic substance of claim 1 in which the fluorinated organic substance is CHF₂Cl.

10. The method of fluorinating a halogenated organic substance of claim 1 in which the introduction of the germanium fluoride compound and the halogenated organic substance is done in the presence of the solvent.

11. A method of fluorinating a halogenated organic substance comprising the steps of:
introducing GeO₂ and UF₄ to CHCl₃ at a temperature between 250 and 320° C. to form CHF₃ and GeCl₄; and
separating the CHF₃ and GeCl₄.

12. The method of fluorinating a halogenated organic substance of claim 11 in which the introduction of the GeO₂ and UF₄ to the CHCl₃ is done in the presence of solvent.

13. A method of fluorinating a halogenated organic substance comprising the steps of:
providing a halogenated organic substance chosen from the group consisting of CHCl₃, CCl₄, C₂Cl₆, and CH₃CCl₃;
introducing a germanium fluoride compound to the halogenated organic substance at a temperature between 250 and 320° C. to increase the fluorine content of the halogenated organic substance, thereby forming a fluorinated organic substance and a halogenated germanium compound; and
separating the fluorinated organic substance and the halogenated germanium compound.

14. The method of fluorinating a halogenated organic substance of claim 13 in which said step of introducing a germanium fluoride compound further includes the step of introducing HF to a germanium fluoride compound to produce GeF₄.

15. The method of fluorinating a halogenated organic substance of claim 13 in which said step of introducing a germanium fluoride compound further includes the step of adding an inorganic fluorine compound to a halogenated germanium compound to increase the fluorine content of the halogenated germanium compound and to form a germanium fluoride compound.

16. The method of fluorinating a halogenated organic substance of claim 13 in which the introduction of the germanium fluoride compound to the halogenated organic substance is done in the presence of a solvent.

17. A method of fluorinating a halogenated organic substance comprising the steps of:
introducing an inorganic fluorine compound to a halogenated, germanium compound to increase the fluorine content of the halogenated germanium compound and to form a germanium fluoride compound;
introducing the germanium fluoride compound to a halogenated organic substance at a temperature between 250 and 320° C. to increase the fluorine content of the halogenated organic substance, thereby forming a fluorinated organic substance and a halogenated germanium compound; and
separating the fluorinated organic substance and the halogenated germanium compound.

18. The method of fluorinating a halogenated organic substance of claim 17 in which the germanium fluoride compound is of the form of RGeFₓ.

19. The method of fluorinating a halogenated organic substance of claim 18 in which the germanium fluoride compound is halogenated.

20. The method of fluorinating a halogenated organic substance of claim 18 in which the germanium fluoride compound is GeF₄.

21. The method of fluorinating a halogenated organic substance of claim 18 in which the germanium fluoride compound includes a halogen or organic substituent.

22. The method of fluorinating a halogenated organic substance of claim 21 in which the organic substituent includes a hydrocarbon, a fluorinated hydrocarbon, or a perfluorocarbon.

23. The method of fluorinating a halogenated organic substance of claim 17 in which the halogenated organic substance is chosen from the group consisting of CHCl₃, CCl₄, C₂Cl₆ and CH₃CCl₃.

24. The method of fluorinating a halogenated organic substance of claim 17 in which the fluorinated organic substance is CHF₃.

25. The method of fluorinating a halogenated organic substance of claim 17 in which the fluorinated organic substance is CHF₂Cl.

26. The method of fluorinating a halogenated organic substance of claim 17 in which the introduction of the germanium fluoride compound and the halogenated organic substance is done in the presence of the solvent.

27. The method of fluorinating a halogenated organic substance of claim 17 in which the inorganic fluorine compound is chosen from the group consisting of HF, F₂, SbF₃, SbF₅, and the transition metal fluorides, including specifically AgF₂, CoF₅, UF₆ and UF₄.

28. The method of fluorinating a halogenated organic substance of claim 15 in which the inorganic fluorine compound is chosen from the group consisting of transition metal oxyfluorides, including specifically UO₂F₂.
